(19) United States
(12) Patent Application Publication
(10) Pub. No.: US 2005/0257789 A1
(43) Pub. Date: Nov. 24, 2005

(54) Foot-activated oxygen flush valve control device and method

(76) Inventor: Maan Salloum, University Place, WA (US)

Correspondence Address: DEAN A. CRAINE, SUITE 140, 400 - 112TH AVENUE NE, BELLEVUE, WA, 98004, US

(21) Appl. No.: 11/113,377

(22) Filed: Apr. 22, 2005

Related U.S. Application Data
(60) Provisional application No. 60/572,359, filed on May 18, 2004.

(51) Int. Cl. A62B 7/00; A62B 9/00; A61M 16/00

(52) U.S. Cl. 128/200.24; 128/204.18

(57) ABSTRACT

A foot-activated, oxygen-flush valve control device disclosed herein that includes a foot-activated switch coupled to a push-button switch assembly mounted on the front surface of the machine and coupled to the anesthesia machine's existing oxygen flush valve control switch. In one embodiment, the foot-activated switch is mechanical and located in a rigid housing located near the floor adjacent to the anesthesiologist's foot. The switch may be connected to a converter mounted on the sides of the anesthesia machine that includes a rod that connects at its distal end to a second mechanical switch located in the converter. A flexible cable extends from the converter to the quick-release button assembly. The button assembly includes an outer cap with a plunger that connects to the distal end of the cable that forces the plunger against the push-button when the foot-activated switch is activated by the user's foot. An intermediate ring selectively holds the outer cap over the existing hub ring on an oxygen flush valve control switch. In another embodiment, the mechanical rods and cables are replaced by a wireless transmitter and receiver disposed between the foot-activated switch and the push-button assembly.

This utility patent application is based on the provisional patent application (Ser. No. 60/572,359) filed on May 18, 2004.

### BACKGROUND OF THE INVENTION

1. Field of the Invention

This invention pertains to foot-activated valves, and more particularly to foot-activated valves used to control an oxygen flush valve on an anesthesia machine.

2. Description of the Related Art

Typical anesthesia machines have a finger controlled, push button switch mounted on its front surface that is coupled to an oxygen flush valve. During administration of anesthesia, an anesthesiologist selectively operates the push button switch to deliberately deliver a large quantity of oxygen to a patient. Typically, the switch is activated during an emergency situation where the normal flow of oxygen delivered to the patient during surgery is insufficient.

Typically, the push button switch opens the flush valve when pressed and automatically closes the valve when released. Because the volume of oxygen delivered through the flush valve is relatively large (50 liters/min), the anesthesiologist typically “taps” and releases the switch to carefully deliver the appropriate amount of oxygen to the patient.

The operating room is set-up so that the anesthesia machine is located at the head of the operating table adjacent to the head of the patient. During administration of anesthesia, the anesthesiologist standing or sitting next to the head of the patient is often required to use two hands to hold a mask tightly against the patient's face and over the mouth and nose so that oxygen is delivered to the patient. In some instances, the anesthesiologist is also required to continuously squeeze the breathing bag to deliver air to the patient. Often another person, such as a technician or nurse, is needed to push the push button switch located on the anesthesia machine that controls the flush valve. When another person is not available, the anesthesiologist has difficulty simultaneously holding the mask against the face, reaching towards the machine, and then manually pressing and releasing the push button switch. Often, several attempts are made before a sufficient amount of oxygen is delivered to the patient.

What is needed is a device that allows an anesthesiologist to easily activate the oxygen flush valve on an anesthesia machine without requiring the anesthesiologist to remove his or her hands from the patient, and without the assistance of another person.

### SUMMARY OF THE INVENTION

It is an object of the present invention to provide a device that allows an anesthesiologist to activate the oxygen flush valve on an anesthesia machine without using his hands and without the assistance of another person.

It is another object of the present invention to provide such a device that includes means to prevent accidental activation.

It is further object of the present invention to provide such a device that can be easily retro-fitted on existing anesthesia machine that uses a manually operated oxygen-flush valve mounted on the front surface of the machine that does not require structural or mechanical alteration or modification of the anesthesia machine, and can be quickly disconnected if needed.

These and other objects of the invention which will become apparent are met by a foot-activated, oxygen-flush valve control device disclosed herein that includes a foot-activated switch connected to a quick-release button assembly mounted over an existing, manually operated push-button switch used to control the oxygen flush valve on an anesthesia machine. The foot-activated switch is located inside a small foot housing located on the floor adjacent to or mounted on the lower corner of the anesthesia machine. The foot housing and the machine are positioned adjacent to the anesthesiologist so that the foot-activated switch may be easily activated by the anesthesiologist's foot while sitting or standing adjacent to the patient. A means for preventing accidental activation of the foot-activated switch during a surgical procedure is also provided on the foot housing.

In the first embodiment, the foot housing contains a mechanical switch that is coupled to a first connecting rod or cable that extends vertically upward from the foot housing to an optional intermediate converter attached at an elevated position on the machine. Located inside the converter is a pivoting arm designed to transfer the longitudinal movement of the rod or cable to a second connecting rod or cable that extends transversely over the front surface of the machine and connects to the quick-release button assembly mounted over the machine's existing push button switch that controls the oxygen flush valve.

In a second embodiment of the device, the converter and second connecting rod or cable are eliminated so that the first connecting rod or cable from the foot housing connects directly to the quick-release button assembly.

In a third embodiment, the foot housing's mechanical switch, the converter, and the connecting rods and cables are replaced with an electrical switch and coil-solenoid located in the foot housing and the quick-release button assembly, respectively. In the third embodiment, the electrical switch and coil/solenoid are electrically connected together with an electrical wire. In still a fourth embodiment, the electrical wires are replaced with a wireless transmitter and a wireless receiver located in the foot housing and outer cap, respectively.

The quick-release button assembly is designed to easily attach and detached to the existing momentary push button switch mounted on the front surface of the anesthesia machine. The quick-release button assembly includes an outer cap that selectively attaches to the hub ring located around the push-button switch. A means for selectively engaging the hub ring is provided for holding the outer cap over the push-button switch. between the hub ring and the outer cap. Located centrally in the outer cap is a secondary plunger that presses against the machine's push button switch.

Using the above described device, a method for delivering a volume of oxygen to a patient using a foot-activated oxygen flush valve on an anesthesia machine during the administration of anesthesia is disclosed.

### DESCRIPTION OF THE DRAWINGS

FIG. 1 is a perspective view of an existing anesthesia machine with a front mounted push-button switch that is connected to the machine's oxygen flush valve with the control device disclosed herein attached to the machine.

FIG. 2 is a perspective view of the control device attached to the push-button switch on the anesthesia machine.

FIG. 3 is a top, sectional view of the quick release button assembly attached to the oxygen flush valve on an anesthesia machine.

FIG. 4 is a side elevational view of the first embodiment of the control device.

FIG. 5 is a sectional side elevational view of the second embodiment of the foot-activated switch located inside the foot housing showing the safety cap in a closed position to prevent accidental activation of the switch.

FIG. 6 is a sectional side elevational view of the foot-activation switch shown in FIG. 5 with the safety cap open and the plunger being forced downward.

FIG. 7 is a side elevational view of the foot activation switch mounted directly on the lower edge the anesthesia machine with the safety cap in a closed position.

FIG. 8 is a side elevational view of the foot-activated switch shown in FIG. 7 with the safety cap moved in an opened position.

FIG. 9 is a sectional, top plan view of the converter mounted on the front surface of the anesthesia machine.

FIG. 10 is a sectional top plan view of the quick-release button assembly mounted over the machine's existing push-button.

FIG. 11 is a section and top plan view of the quick-release button assembly being removed from the machines's existing push-button.

FIG. 12 is a sectional side elevational view of another embodiment of the foot housing with an electrical switch located therein that is electrically connected via an electrical wire to a coil-solenoid located in the outer cap of the quick-release button assembly.

FIG. 13 is a sectional side elevational view of another foot-activation switch in which a wireless transmitter and a wireless receiver are used in place of the electrical wire.

### DESCRIPTION OF THE PREFERRED EMBODIMENT(S)

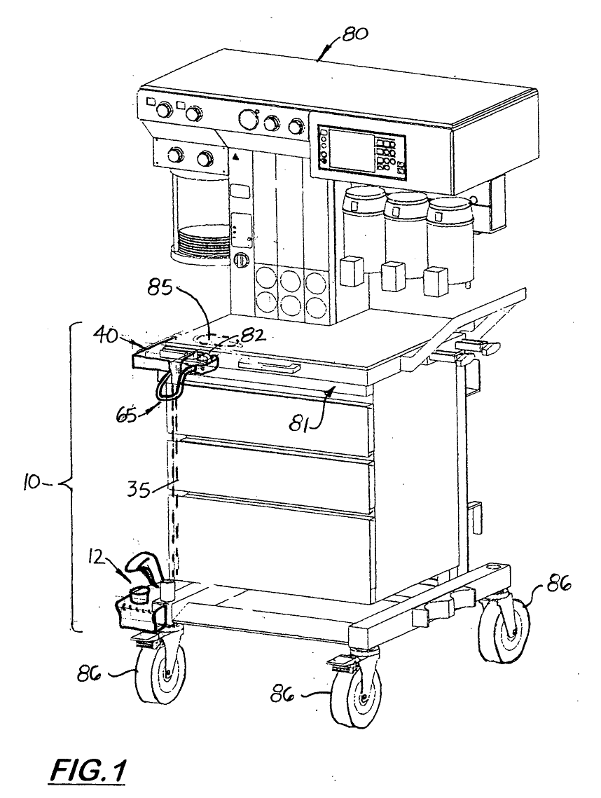

Referring to FIG. 1 there is shown and described a foot activated, oxygen-flush valve control device 10 disclosed herein designed to be used with a standard anesthesia machine 80 with a front mounted, push-button switch 82 that operates the machine's oxygen flush control valve 85. A typical anesthesia machine 80 is mounted on caster wheels 86 with a push-button switch 82 positioned on its front panel 81 within easy reach of the anesthesiologist sitting or standing next to the patient's head during a surgical proceeding. The push-button switch 82 is located inside a hub ring 83 that is separately attached to or integrally formed on the front panel 81 of the machine 80. The purpose of the device 10 is to allow an anesthesiologist to activate the switch 82 using his or her foot 98 so that both hands may be used or maintained on the patient.

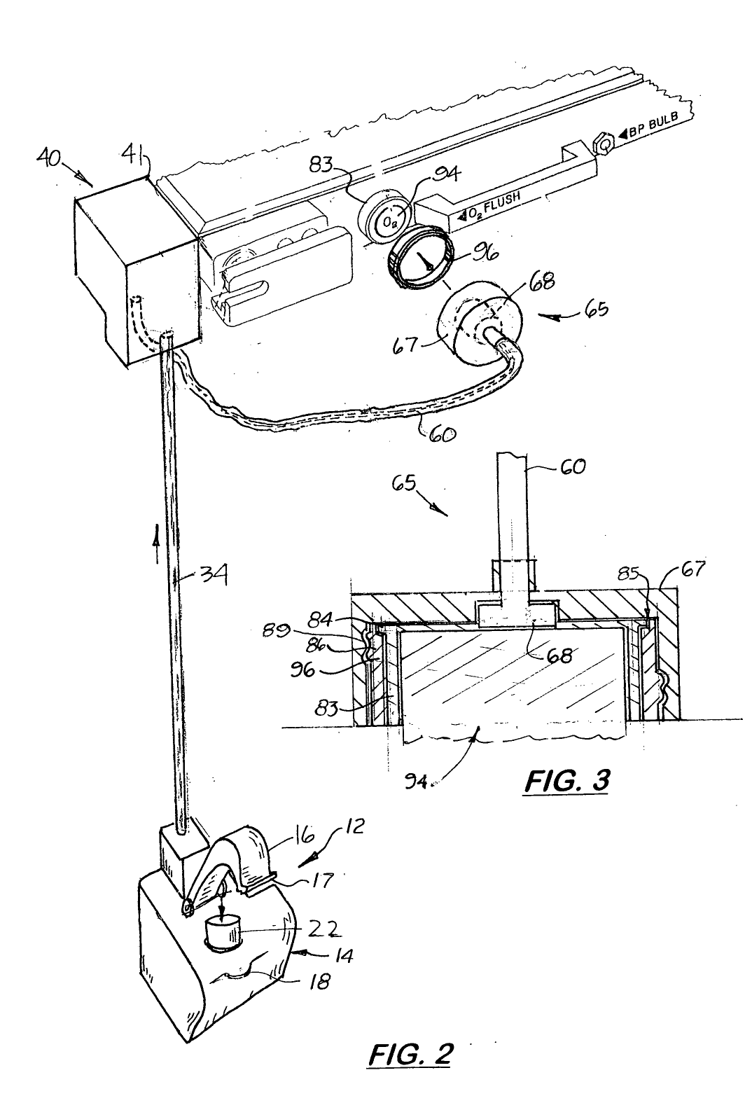

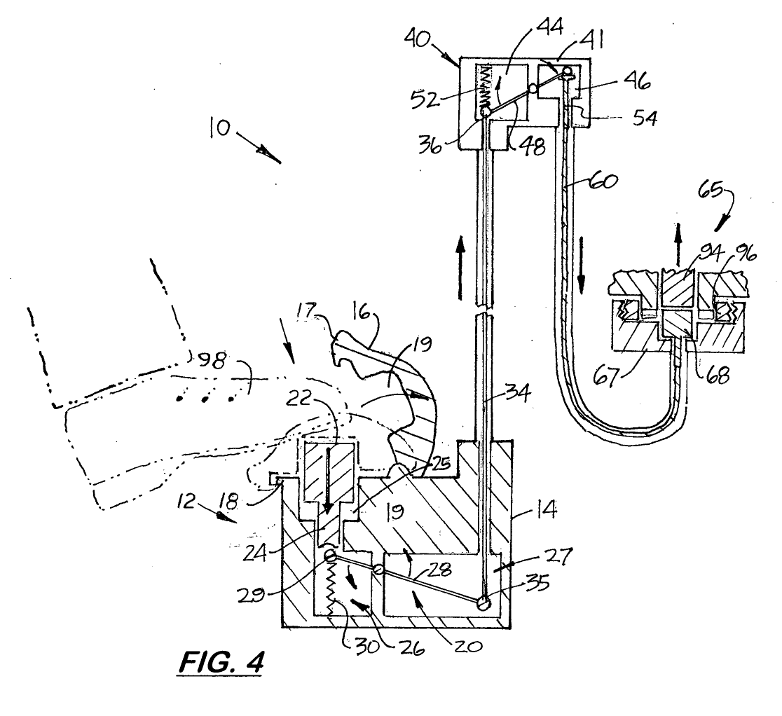

The device 10 includes a foot-activated switch 12 positioned on the floor or directly attached to the lower corner of the machine 80 just above the caster wheel 86. FIGS. 2 and 4 show the first embodiment of the switch 12 that includes rigid housing 14 that can be placed either on the ground or securely mounted to the corner of the machine 80 with suitable connectors (not shown). The rigid housing 14 includes a pivoting cover 16 that covers a plunger 22 located therein. As shown in FIG. 4, formed inside the rigid housing 14 is a first mechanical pivoting switch, generally designated 20, designed to couple the longitudinal movement of the foot plunger 22 to the proximal end of a first connecting rod/cable 34 that extends vertically upward along the side of the machine 80. The removable cover 16 is pivotally connected to two ears located on the top surface of the rigid housing 14. Formed on the front edge of the cover 16 is a recessed lip 17 designed to engage a forward extending lip 18 formed on the front surface of the rigid housing 14 to temporarily hold the cover 16 in a closed position on the rigid housing 14. During use, the lip 17 on the cover 16 is lifted by the anesthesiologist's foot 98 to pivot the cover 16 upward and rearward to expose the foot plunger 22, as shown in FIG. 4.

Formed on the inside surface of the cover 16 is an upper ward extending cavity 19 designed to receive the cylindrical upper section of the plunger 22. Integrally formed on the plunger 22 is a lower narrow neck 24 that fits inside complimentary shaped cavities 25, 26, respectively. Located inside the rigid housing 14 and adjacent to the lower cavity 26 is a main cavity 27. Extending between the lower cavity 26 and the main cavity 27 is a pivoting arm 28. The first end 29 of the pivoting arm 28 is biased in an upward position by a spring 30 located inside the lower cavity 26. The second end 31 of the pivoting arm 28 extends into the main cavity 27 and connects to the proximal end of the first connecting rod/cable 35.

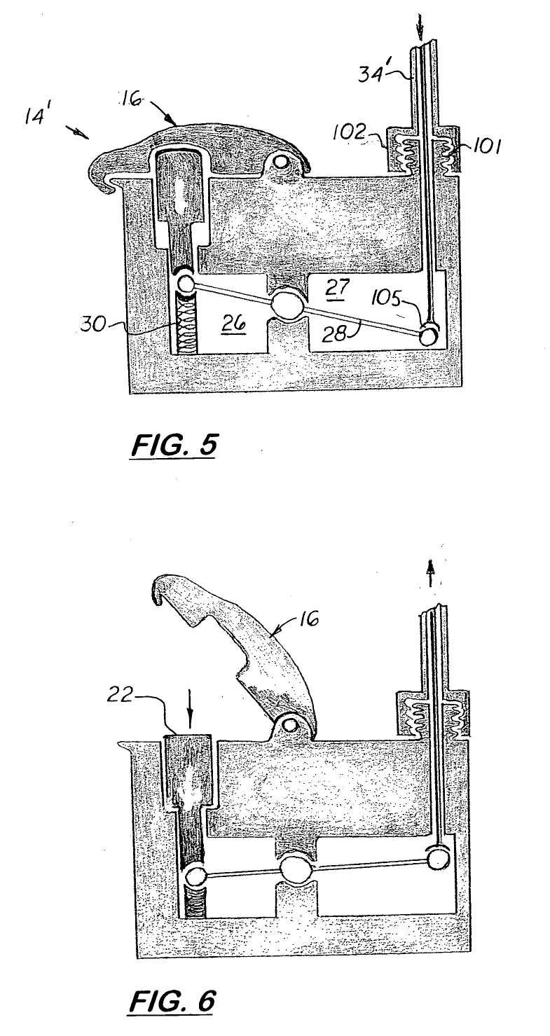

FIGS. 5 and 6 shown an alternative footing housing 14′ that uses a modified first connecting rod/cable 35′ with a threaded end cap 102 that connects to a threaded neck 101 formed on the foot housing 14′. This embodiment is useful, because it allows the foot housing 14′ to easily connected and disconnected from the first connecting rod or cable 35′.

Referring again to FIG. 4, attached to the sides of the machine 80 directly above the foot switch 12 is a converter 40 used to transmit the up and down movement or side to side movement of the first connecting rod/cable 34 into a pushing movement by the second connecting rod or cable 60 that extends from the opposite end of the converter 40. In the first embodiment, the converter 40 includes a rigid housing 41 attached to the side of the machine 80 with suitable connectors (not shown). The rigid housing 41 includes a larger main cavity 44 and an adjacent smaller second cavity 46. Disposed between the main cavity 44 and second cavity 46 is a second pivoting arm 48. A second spring 52 located inside the main cavity 44 is used to bias the proximal end 49 of the second pivoting arm 48 in a downward position inside the main cavity 44. Attached to the distal end of the connecting rod 34 is a ball 36 that presses against the spring 52. The distal end 50 of the second pivoting arm 48 is located adjacent to a plunger cap 54 is attached to the proximal end of a flexible cable 60 that extends into the second cavity 46. During operation, the plunger cap 54 moves up and down inside the second cavity 46 in response to the up and down movement of the first connecting rod/cable 34. As the plunger cap 54 moves up and down inside the second cavity 46, the second connecting rod/cable 60 extends and retreats into the rigid housing 41.

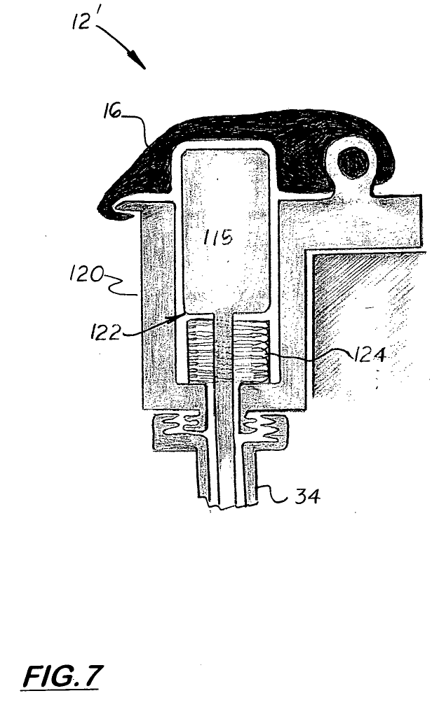

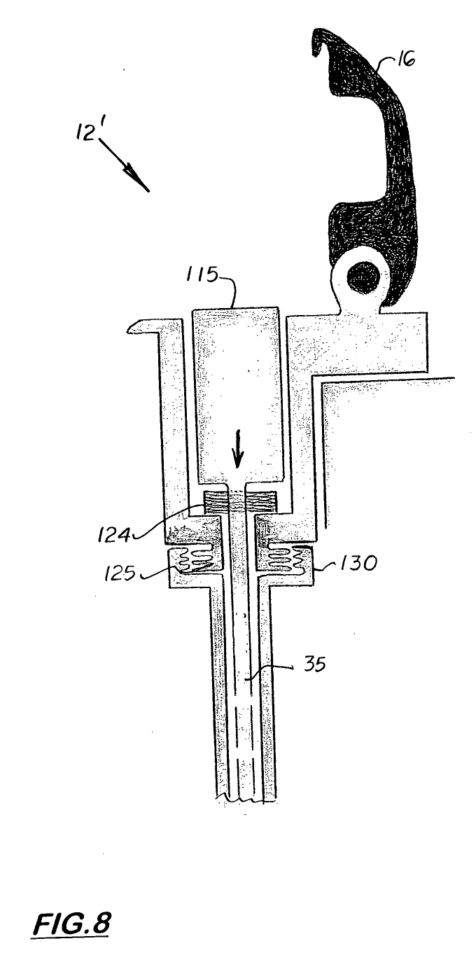

FIGS. 7 and 8 shows another embodiment of the foot activation switch 12′ in which the pivoting arm 28 used on the first activation switch 12 is eliminated and the plunger 115 is directly connected to the distal end of the first connecting rod/cable 34. The housing 120 for the switch includes a cylindrical cavity 122 with a narrow cable opening 123 formed on one end. The plunger 115 is smaller in diameter than the cavity 122 and moves longitudinally and freely therein. A spring 124 is disposed between the inside surface of the cavity 122 and the end of the plunger 115 thereby biasing the plunger 115 outward from the housing 120. Formed on the outer edge of the housing 120 opposite the plunger 115 is a threaded neck 125 that connects to a threaded collar 130 attached to the first connecting rod/cable 34. Like the first embodiment, a pivoting safety cap 16 that locks over the plunger 115 that can be opened and moved to an open position by the foot is also provided.

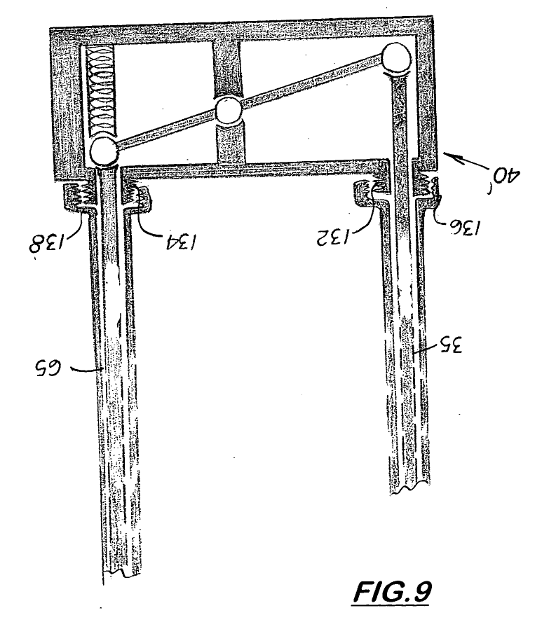

FIG. 9 shows an alternative converter 40′ with two parallel threaded necks 132, 134 that connect to threaded ends caps 132, 134 on the ends of the first and second connecting rods/cables 35, 65, respectively. The alternative converter 40′ is designed to be easily disconnected from the connecting rods/cables 35, 65 so that the device may be easily installed on an existing machine 80.

FIGS. 2, 3 and 4 shows the quick-release button assembly 65 comprising an outer cap 67 that include an internal thread that selectively attaches to the external thread on an intermediate ring 96 that engages the existing hub ring 83 around a push-button switch 94 connected to the machine flush valve. The existing hub ring 83 includes an upper lip 84 that engages a recessed edge 85 formed near the upper edge of the surrounding intermediate ring 96. Formed on the outer surface of the intermediate ring 96 are external threads 86 that engage internal threads 89 on the outer cap 67. During assembly, the intermediate ring 96 attaches to the hub ring 83 and the outer cap 67 attaches to the intermediate ring 96. Located centrally on the outer cap 67 is a plunger 68 that connects to the distal end of the second connecting rod/cable 65. During assembly, the outer cap 67 is aligned over the hub ring 96 so that the plunger 68 presses against the button 94.

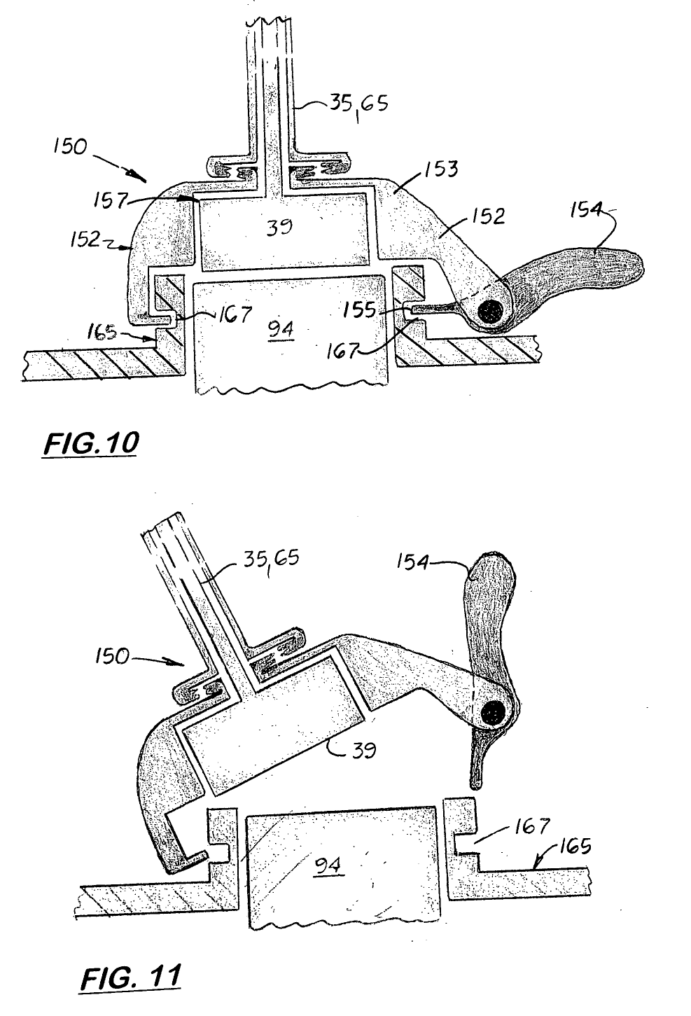

FIGS. 10 and 11 show an alternative quick-release button assembly 150 that includes a clamp-style outer cap 152 that engage an existing hub ring 165 with at least two opposite holes formed therein. The outer cap 152 includes an inward lip and an opposite lever arm 154 with an inward directed tip 155. The lip engages one hole on the hub ring 165 and the tip 155 engages the opposite hole. The lever arm 154 is attached to a diagonal member 152 that extends downward from the outer cap's cylindrical shaped main body 151. Formed inside the main body 151 is an end cap cavity 157 designed to receive a complimentary shaped end cap 39 attached to the distal end of the first or second connecting rod/cable 35, 65, respectively. Formed on the outer surface of the outer cap 152 is a threaded neck 158 that receives the threaded end cap on the first or second connecting rod/cable 35, 65, respectfully.

The first and second connecting rods/cables 35, 65 may be made of rigid or flexible material. In the preferred embodiment, the connecting rods/cables 35, 65 are covered by protective sheathing.

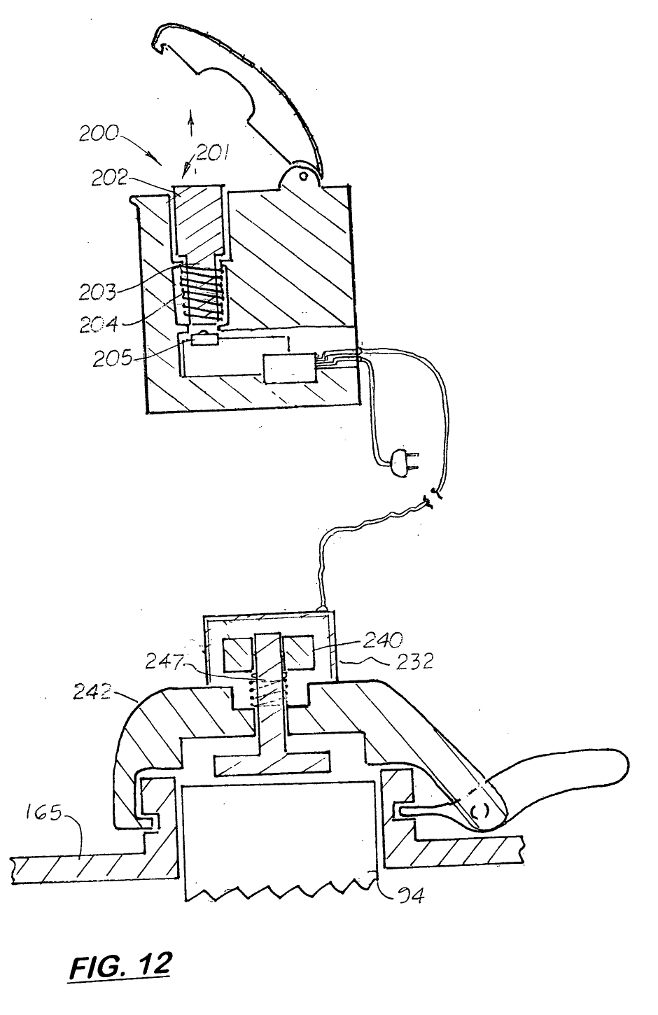

FIG. 12 is an illustration of the third embodiment of the device in which the pivoting switch and first connecting rods/cable 35, the second connecting rod 65 and the converted 40 are all eliminated and replaced with an electrical switch 210 located inside the foot housing 14 and coil/solenoid located in the outer cap 242 of the quick-release button assembly 230. The foot housing 14 includes a plunger 201 with a wide upper cylindrical body 202 and a lower narrow neck 203. Disposed around the neck 203 is a spring 204. Attached to the distal end of the plunger 201 is an electrical contact 205 that completes a simple electrical circuit with a contact 206 located on the bottom surface of the plunger 201. When the plunger 201 is pressed downward, the electrical circuit is completed that produces an electrical signal that is transmitted via an electrical wire 212 to a coil-solenoid 240 located in the outer cap 240 of the quick-release button assembly 235.

The outer cap 240 selectively attaches directly to the hub ring 83 disposed around the oxygen valve switch 94. The coil-solenoid 240 includes a T-shaped plunger 246. When the coil-solenoid 240 receives an electrical signal from the electrical switch 210 in the foot housing 14, the plunger 246 is forced outward and presses against the switch 94 to activate the oxygen valve. A spring 248 is disposed around the center post of the plunger 246 so that the plunger 246 automatically returns to its resting position.

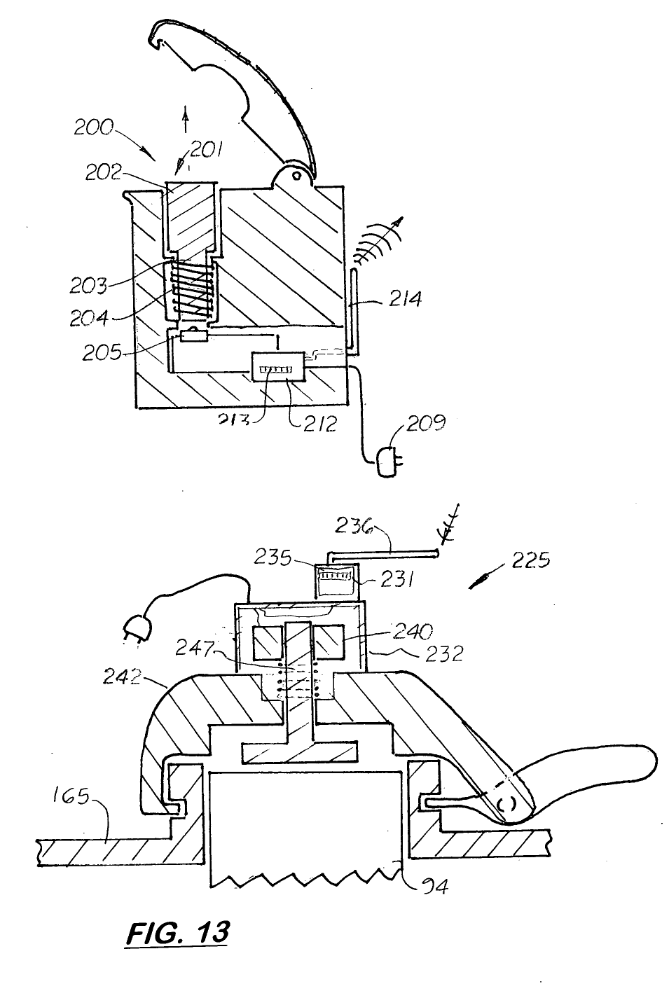

FIG. 13 is an illustration of a third embodiment of the device in which the wire is replaced with a wireless transmitter 212. The switch 200 includes the same plunger 201 with a wide upper cylindrical body 202 and a lower narrow neck 203. Disposed around the neck 203 is a spring 204 designed to apply an upward biasing pressure against the plunger 201. Located below the distal end of the neck 203 is a contact switch 205 electrically connected to a wireless transmitter 212. An antenna 214 is mounted on the side of the housing 207 an external electrical power source 209 is used to supply electricity to the contact switch 205 and the wireless transmitter 212. It should be understood however, that a battery could be used in place of the external electrical power source 209. During use, when the anesthesiologist applies pressure with his or her foot on the plunger 201, the distal end of the plunger 202 makes contact with the contact switch 205, which in turn, activates the wireless transmitter 212.

Also shown in FIG. 13, the quick-release button assembly 225 is mounted over the machine's existing push-button switch 94 includes a wireless receiver 235 and an electrical coil solenoid switch 240 mounted therein. The coil solenoid 240 is mounted inside the outer cap 242 that selectively attaches to the hub ring 83. When the coil solenoid 240 is activated, the T-shaped plunger 247 moves downward through the coil solenoid 240 and presses against the quick-release button switch 94. Attached to the outer cap 242 is a small housing 232 in which the coil solenoid 240 is located. Attached to the wireless receiver 234 is an antenna 236 design to receive the radio signals from the wireless transmitter 212.

Attached to the wireless transmitter 212 and receiver 235 are optional frequency adjustment switches 213, 231, respectively, each with a plurality of slide-style dip switches that enable the signal frequency transmitted between the transmitter 212 and receiver 235 to be adjusted to avoid interference with other medical devices. In the preferred embodiment, the transmitter 212 and receiver 235 use a radio signal 280 with a frequency between 480 and 490 mhz.

During use, both the wireless transmitter and the wireless receiver 234 are electrically energized. When the wireless receiver 234 receives a signal 280 from the transmitter 212, the coil solenoid 240 is activated which forces the plunger 247 downward and against the switch 94. When the signal 280 is discontinued, the polarity of the coil solenoid 240 is reversed and the plunger 247 returns to its original position.

The above-described device has very few moving parts or components that require little or no maintenance. Since industry standards require that anesthesia machines be inspected every three to four months, inspection and operation of the device can be easily carried out at the same time.

Using the above described control device 10, a method for quickly delivering a large volume of oxygen to a patient during the administration of anesthesia comprising the following steps:
a. selecting an anesthesia machine 80 with a manually operated push-button switch 94 coupled to an oxygen flush valve;
b. selecting a foot-activated oxygen flush valve control device 10 that includes a foot-activated switch 12 with a removable cover 16, a first mechanical switch 20 located inside a rigid housing 14, a quick-release button assembly 60 mounted over the push-button switch 94, means for coupling the movement of the first mechanical switch 20 inside said foot-activated switch to said quick-release button assembly 60 whereby when said foot-activated switch 12 is activated, the push-button switch 94 on said anesthesia machine 80;
c. mounting said quick-release button over said manually operated switch on said anesthesia machine 80;
d. positioning said foot-activated switch 12;
e. opening said removable cover 16; and,
f. activating said foot-activated switch 12 to press said push-button switch 94 on said anesthesia machine 80 and activate said oxygen flush valve.

In compliance with the statute, the invention described herein has been described in language more or less specific as to structural features. It should be understood, however, that the invention is not limited to the specific features shown, since the means and construction shown, is comprised only of the preferred embodiments for putting the invention into effect. The invention is therefore claimed in any of its forms or modifications within the legitimate and valid scope of the amended claims, appropriately interpreted in accordance with the doctrine of equivalents.

1. A foot-activated, oxygen flush valve control device used with an anesthesia machine with a manually operated push button oxygen flush valve switch, said device comprising:
a. a foot-activated switch;
b. a quick-release button assembly mounted over the push-button oxygen flush valve switch, said quick-release button assembly including a plunger that presses against said valve switch to activate said valve switch and means for holding the button assembly over the existing push button oxygen flush valve switch on an anesthesia machine; and,
c. means for coupling the movement of said foot-activated switch to said plunger in said quick-release button assembly.

2. The foot-activated oxygen flush valve control device as recited in claim 1, wherein said means for coupling the movement of said foot-activated switch to said plunger in said quick release button assembly is a connecting cable that extends between said foot-activated switch and said plunger.

3. The foot-activated oxygen flush valve control device as recited in claim 1, wherein said means for coupling the movement of said foot-activation switch to said plunger in said quick release button assembly is a vertically aligned connecting rod that connects to a converter located on said anesthesia machine and a second connecting rod that connects said plunger in said quick release button assembly.

4. The foot-activated oxygen flush valve control device as recited in claim 1, wherein said means for engaging the existing push button oxygen flush valve switch is an outer cap and a hub ring that is placed around said valve switch.

5. The foot-activated oxygen flush valve control device is recited in claim 1, wherein said means for engaging the existing push button oxygen flush valve is an outer cap with a inward extending lip structure and a lever that engages the sides of a activation switch.

6. The foot-activated oxygen flush valve control device as recited in claim 1, further including a means for preventing accidental activation by the user's foot.

7. The foot-activated oxygen flush valve control device as recited in claim 4, further including a means for preventing accidental activation by the user's foot.

8. The foot-activated oxygen flush valve control device, as recited in claim 6, wherein said means for preventing accidental activation is a removable cover on said foot-activation switch.

9. The foot-activated oxygen flush valve control device as recited in claim 1, wherein said foot activated switch is attached to said anesthesia machine.

10. The foot-activated oxygen flush valve control device as recited in claim 2, further including means for preventing accidental activation by the user's foot.

11. The foot-activated oxygen flush valve control device, as recited in claim 8, wherein said means for preventing accidental activation is a removable cover on said foot-activation switch.

12. The foot-activated oxygen flush valve control device as recited in claim 1, wherein said foot activated switch is attached to said anesthesia machine.

13. The foot-activated oxygen flush valve control device as recited in claim 3, further including means for preventing accidental activation by the user's foot.

14. The foot-activated oxygen flush valve control device, as recited in claim 11, wherein said means for preventing accidental activation is a removable cover on said foot-activation switch.

15. The foot-activated oxygen flush valve control device as recited in claim 1, wherein said means for coupling the movement of said foot-activated switch to said plunger in said quick release button is an electrical solenoid coupled to said plunger in said foot housing, a wireless transmitter coupled to said foot-activation switch, and a wireless receiver coupled to said solenoid, said wireless transmitter and said wireless receiver capable of communicating a signal to couple the activation of said foot-activation switch with the solenoid to activate the plunger on said oxygen flush valve.

16. The foot-activated oxygen flush valve control device, as recited in claim 15, wherein said wireless transmitter and said wireless receiver is a radio transmitter and a radio receiver, respectively.

17. The foot-activated oxygen flush valve control device, as recited in claim 15, wherein said radio transmitter and said radio receiver communicate with a signal compatible with the medial equipment located in an operating room.

18. A method for quickly delivering a large volume of oxygen to a patient during the administration of anesthesia comprising the following steps:
a. selecting an anesthesia machine 80 with a manually operated push-button switch 94 coupled to an oxygen flush valve;
b. selecting a foot-activated oxygen flush valve control device 10 that includes a foot-activated switch 12 with a removable cover 16, a first mechanical switch 20 located inside a rigid housing 14, a quick-release button assembly 60 mounted over the push-button switch 94, means for coupling the movement of the first mechanical switch 20 inside said foot-activated switch to said quick-release button assembly 60 whereby when said foot-activated switch 12 is activated, the push-button switch 94 on said anesthesia machine 80;
c. mounting said quick-release button over said manually operated switch on said anesthesia machine 80;
d. positioning said foot-activated switch 12 next to the foot of an anesthesiologist providing anesthesia during a surgical procedure;
e. opening said removable cover 16 to provide access to said plunger on said foot activate switch; and,
f. activating said foot-activated switch 12 with the anesthesiologist's foot to press said push-button switch 94 on said anesthesia machine 80 and activate said oxygen flush valve.
